(19) United States
(12) Patent Application Publication
(10) Pub. No.: US 2009/0311304 A1
(43) Pub. Date: Dec. 17, 2009

(54) DRUG-LOADED IMPLANT

(76) Inventors: Alexander Borck, Aurachtal (DE); Tobias Diener, Erlangen (DE); Claus Harder, Uttenreuth (DE); Michael Tittelbach, Neurnberg (DE); Amir Fargahi, Buelach (CH)

Correspondence Address: GREER, BURNS & CRAIN, 300 S WACKER DR, 25TH FLOOR, CHICAGO, IL, 60606, US

(21) Appl. No.: 12/482,929

(22) Filed: Jun. 11, 2009

(30) Foreign Application Priority Data
Jun. 12, 2008 (DE) 10 2008 002 395.7

(51) Int. Cl. A61L 27/54

(52) U.S. Cl. 424/426

(57) ABSTRACT

The invention relates to a drug-loaded implant having a carrier body (10) and at least one drug for delivery into a delivery region. The drug is provided for chemotherapy and/or palliative treatment and can be released topically and/or regionally in a controlled manner at a predefinable rate and/or over a predefinable period of time in the intended active state of the implant.

### FIELD

The invention relates to a drug-loaded implant according to the preamble of Patent claim 1.

### BACKGROUND

In the field of topical and/or regional therapy, one or more drugs must be administered in high doses without inducing any negative side effects in the surrounding tissue or body regions outside of the target region due to the mechanism of action and/or the dose of the medication.

Medicinal treatment and/or palliative treatment of cancerous tissue requires high-dose administration of cytostatic pharmaceutical drugs and/or opiates, for example. The adverse effects on healthy tissue regions or organs associated with oral or intravenous administration are not insignificant and contribute toward increased morbidity of patients.

In inoperable tumor diseases, in particular HCC (hepatocellular carcinoma), various treatments are used, but all of them are associated with major disadvantages. Known examples include transarterial chemo-embolization (TACE), hepatic arterial infusion (HAI), cryotherapy, laser-induced thermal therapy (LITT), radiofrequency ablation (RFA), percutaneous ethanol injection (PEI). Large portions of healthy liver tissue may be damaged through TACE in particular, thus resulting in a lack of reserve hepatic function. The treatments are used to bridge the waiting time until a liver transplant. There is no curative effect.

The doses in administration of single doses of drugs are often inadequate; in particular the drug concentration often drops below the therapeutic window too rapidly. Systemic administration of cytostatics is also impossible with liver tumors because of the patient's general health, which results in aftertreatments and prolonged hospitalization for the patient. Unexamined Patent US 2002/0133224 A1 discloses a stent surrounded by a microporous polymer membrane in which a pharmaceutical drug may be embedded. U.S. Pat. No. 7,056,339 B2 discloses a stent with a drug-loaded matrix in abluminal and adluminal channels at the surface of the stent. The drug is contained in microspheres. The outside of the stent is surrounded by a covalently bonded gel. The drug may be delivered over a long period of time.

### OBJECTS

The object of the invention is to create a drug-loaded implant that provides high doses of a drug which cannot be used at all or not in a comparable potency in systemic administration due to the type and/or efficacy.

This object is achieved according to the invention by the features of Patent claim 1. Advantageous embodiments and advantages of the invention are derived from the additional claims and the description.

### SUMMARY

A drug-loaded implant having a carrier body and at least one drug for delivery in a delivery region is proposed, in which the drug is provided for chemotherapy and/or for palliative treatment and can be released in the active state of the implant when used as intended in the body of a living creature, where it can be released topically and/or regionally in a controlled manner at a predefined rate and/or over a predefined period of time. Depending on the embodiment, the implant may be provided for use in a blood vessel or for use in tissue in the body. It is advantageously possible to administer the drug(s) in a high dose topically in a targeted manner without inducing adverse negative effects in the surrounding tissue or in the surrounding regions of the body due to the mechanism of action and/or the size of the dose of the medication. Furthermore, it is possible to create an implant with a very high drug content. A high drug content usually has a very negative effect on mechanical stability with the known implants.

The term “drug” in the present context refers in general to a single drug, a mixture of drugs or a drug formulation and/or another material that is provided for release in a targeted manner, advantageously with a pharmaceutical and/or biological potency. The drug-loaded implant may advantageously be loaded with cytostatic drugs which would lead to severe adverse effects if administered systemically, e.g., by oral ingestion due to its nature and/or its potency. Topical administration of the drug makes it possible for doses which could not be achieved systemically with a comparable potency to be delivered to a treatment site. The implant may thus be introduced into a blood vessel a few centimeters upstream from a tumor, such that the drug is conveyed by the bloodstream to the tumor. In addition, the delivery of the drug may take place in a controlled manner, so that the drug is made available over a sufficiently long period of time.

Delivery of the drug may advantageously be based on the desired benchmark values. The most homogeneous possible release of the drug over a longer period of time, e.g., two weeks or more, can be achieved in this way. A dose peak may be achieved relatively quickly, e.g., after 24 hours at the latest. The decline in dose after the end of the treatment period can be adjusted in a suitable manner, but is preferably adjusted to be as steep as possible. Depending on the design of the implant, the release may be accomplished by elution or diffusion. It is also conceivable to adjust a desired relatively low drug level in the blood systemically, superimposed on a short-term peak of the inventive implant.

It is especially advantageous when the carrier body can be designed to be biodegradable. Then removal of the carrier body after the end of the treatment period may be suppressed. The carrier body may advantageously be formed from a material that is degraded at a sufficiently slow rate, in particular more slowly than the release of the drug. Carrier bodies, which release embolization particles as the drug, for example, and which carry one or more medications for release in the interior, for example, may also be used.

According to a preferred embodiment, the drug may be embedded in a polymer matrix from which the drug can be released, e.g., by degradation of the polymer matrix and/or by diffusion. If the carrier body is a stent, for example, then the individual struts of the stent may be surrounded by the polymer matrix while interspaces in the sent remain open. The carrier body may advantageously be coated with the polymer matrix in at least some areas.

Alternatively, the polymer matrix may surround the carrier body in the manner of a membrane or sheathing. If the carrier body is a stent, for example, then both the struts and the interspaces between the struts of the stent are covered by the polymer matrix abluminally on the outside circumference and/or (ad)luminally on the inside circumference. A release that is variable over time can be made possible through the use of degradable and/or absorbable polymers having different degradation rates.

The drug may advantageously be arranged in recesses and/or cavities in the carrier body. An especially large amount of drug may be deposited there. The drug may preferably be released by elution or diffusion. However, as an alternative or in addition, it is also possible to arrange the drug in the recesses and/or cavities in a polymer matrix. In this case, the drug may be released with a time lag. A release that is variable over time can be made possible through the use of degradable and/or absorbable polymers having different degradation rates.

The polymer matrix may advantageously form the carrier body in at least some areas. Such a self-supporting polymer matrix may be designed as a tube or a pen, for example.

The drug may advantageously be arranged between two polymer layers which surround the hollow carrier body adluminally and abluminally. The drug may advantageously be released, e.g., by dilution or diffusion. The rate of release can be adjusted easily by defining channel geometries, e.g., in or between the polymer layers.

Alternatively, the carrier body may be formed by a tube, which is inverted in some areas, so that the drug is arranged in the area of the cavity formed by the inversion between the inner tubular section and the inverted tubular section arranged over the former. The tube allows easy production with good mechanical properties.

For treatment of special diseases of the liver, embolization particles to be released in a polymer matrix may advantageously be provided so that they seal a blood vessel containing the implant in a targeted manner before or after a preferably topical or regional drug delivery. The polymer and/or the embolization particles may preferably be loaded with the drug.

A drug-impermeable cover layer may be provided on the implant protecting the abluminal tissue of the delivery region from the drug in the implanted state. The drug-impermeable cover layer may be designed to be permanent and/or nonabsorbable or it may be made of absorbable material which has a much lower degradation rate than the drug release rate. The drug may thus be released reliably in a predetermined manner before the carrier body is degraded.

The carrier body may advantageously be designed as a stent, preferably as a self-expanding stent. The carrier body may alternatively be designed as a tube or a rod in the form of a so-called drug delivery pen. As a tube, the carrier body may preferably be a hollow tube, where the tube wall is loaded with the drug. This may be embedded in a polymer matrix, for example, which is arranged on the tube wall, or it may be arranged in cavities or recesses in the tube wall, which may then be designed to be porous or roughened, for example, accordingly. As a rod, the carrier body preferably has a core that contains the drug and from which the drug can be released through a suitably permeable rod wall and/or from one or both end faces of the core. Such a design in the form of a drug delivery pen is advantageous for introduction into the tissue. This yields a relatively stable and load-bearing device.

When the inventive implant is provided for introduction into a blood vessel and can be affixed in or on the vascular wall, it need not have as great a supporting force as a stent, for example. The design may advantageously be such that more drug can be stored at the expense of the supporting force.

According to another advantageous possibility, the carrier body may be formed by a rolled film having recesses to receive the drug. In addition or alternatively, the carrier body may be formed from a rolled or slotted film. The film may be formed from a permanent material, preferably a nickel-titanium alloy (nitinol) or from a biodegradable material.

The invention is explained in greater detail below on the basis of exemplary embodiments illustrated in the drawings as examples. They show in schematic diagrams:

### BRIEF DESCRIPTION OF THE DRAWINGS

FIG. 1 a cross section through a preferred implant according to a preferred first embodiment of the invention;

FIGS. 2a-2d various versions of a preferred implant in the form of the drug delivery pen according to one embodiment of the invention for introduction preferably into tissue;

FIG. 3 a preferred insertion system for a preferred implant according to the invention as illustrated in FIGS. 2a-2d;

FIGS. 4a-4c various view of a preferred implant according to another preferred embodiment of the invention having a carrier body, which is arranged between two polymer layers;

FIGS. 5a, 5b a drug-loaded polymer matrix (FIG. 5a) and a carrier body having a drug-loaded polymer matrix (FIG. 5b) according to another preferred embodiment of the invention;

FIG. 6 a view of a carrier body formed from a polymer matrix according to another embodiment of the invention;

FIGS. 7a, 7b a longitudinal section through a preferred implant according to another preferred embodiment of the invention (FIG. 7a) and a cross section through a variant of the implant (FIG. 7b);

FIGS. 8a, 8b a preferred carrier body (FIG. 8a) and an installation situation of the carrier body (FIG. 8b); and

FIGS. 9a, 9b a preferred carrier body in the rolled state (FIG. 9a) and in the unrolled state (FIG. 9b) according to another embodiment of the invention.

### DETAILED DESCRIPTION

Elements that are functionally the same or have the same effect are each labeled with the same reference numerals in the figures. The figures show schematic diagrams of the invention. They illustrate nonspecific parameters of the invention. In addition, the figures show only typical embodiments of the invention and should not restrict the invention to the embodiments depicted here.

An inventive drug-loaded implant comprises a carrier body and an drug for release in a delivery region, whereby the drug is provided for chemotherapy and/or palliative treatment and can be released topically and/or regionally in the intended active state of the implant in a body of a living creature in a controlled manner and a predefinable rate and/or over a predefinable period of time. Depending on the embodiment, the carrier body may be provided for insertion into a blood vessel or into tissue. The carrier body may be coated or covered with a polymer or may consist of a polymer which is biodegradable and dissolves in the body. If necessary, the implant may also comprise a nondegradable material.

Preferred drugs for use in pure form or incorporated into a polymer matrix include in particular medications that are suitable specifically for chemotherapy or for palliative treatment of cancer. Furthermore, one or more of the drugs may be selected from the groups of (some of the following terms may denote brand names):
Immunosuppressants (e.g., sirolimus),
calcineurin inhibitors (e.g., tacrolimus),
antiphlogistics (e.g., cortisone, diclofenac),
anti-inflammatories (e.g., imidazole, pimecrolimus),
steroids,
proteins/peptides,
   in particular one or more drugs for chemotherapy or palliative treatment of cancer, e.g.,
   105AD7
   13-cis RA
   17-AAG
   1A7
   2A11
   3F8
   3H1
   5-fluorouracil
   9-cis-retinoic acid
   A10/AS2-1
   ABT-510
   ABX-EGF
   Adp53
   Adriamycin PFS
   Adriamycin RDF
   Alemtuzumab
   Alitretinoin
   Allovectin-7
   Altretamine
   Amifostine
   Angiostatin
   Angiozyme
   Antineoplastons
   Anti-Tac-PE38 (LMB-2)
   AP12009
   Aplidine
   Apomine
   Aromasin
   Arsenic trioxide
   Astrasentan
   Azathioprine
   Bay 43-9006
   Bay 50-4798
   Bay 12-9566
   BB-10010
   BB-10901
   BEC2
   Bevacizumab
   Bexarotene
   Bicalutamide
   BL22
   BMS-214662
   BMS-247550
   BMS-275291
   BNP7787
   Bryostatin-1
   Busulfan
   Busulfex
   Buthionine sulfoximine
   Capecitabine
   Carboxyamidotriazole
   Casodex
   CC-5013
   CCI-779
   Celecoxib
   Cetuximab (Erbitux)
   Ch14.18
   CHS828
   CI-1040
   CI-994
   Cisplatin
   Clodronate
   CM101
   COL-3
   Combretastatin A4
   CP-461
   CP-471,358
   CP-547,632
   CT 2584
   Cyclophosphamide
   Cyclosporin A
   Cytoxan
   Decitabine
   Denileukin diftitox (ONTAK)
   Depsipeptide
   Dexniguldipine
   Dexrazoxane
   Dexverapamil
   Dolastatin-10
   Doxorubicin
   DPPE
   EMD 273063
   EMD 55900
   EMD 72000
   Endostatin
   Enzyme L-asparaginase
   EP0906
   Epirubicin
   Epratuzumab
   ET-743
   Exemestane
   Exisulind
   FB642
   Femara
   Fenretinide
   Finasteride
   FK317
   FK866
   Flavopiridol
   G17DT
   GBC-590
   GD0039
   GEM231
   Gemtuzumabozogamicin
   Genasense (Genta)
   GF120918
   GM-CSF
   GW572016
   H22xKI-4
   Hexalen
   HSV-TK VPC
   HuM195
   HuMV833
   ICR62
   IL13-PE38QQR
   IL-2/histamine
   Ilmofosine
   ILX23-7553
   IM862
   IMC-1C11
   Imuran
   ING-1
   Interleukin-12
   INX3280
   Irofulven
   ISIS-2503
   ISIS-3521
   ISIS-5132
   J591
   Kahalalide F
   KM871
   KW-2189
   L-778 123
   LAF389
   LAK, TIL, CTL
   LErafAON
   Letrozole
   Lobradimil
   Lovastatin
   LU103793
   LY-293111
   LY-317615
   LY-335979
   LY-355703
   Lyprinol
   Marimastat
   MCC-465
   MDX-010
   MDX-11
   MDX-447
   MDX-H210
   Melatonin
   Methotrexate
   MG98
   Mifeprex
   Mifepristone
   Mitoxantrone
   MM1270
   MS209
   Myleran
   Mylotarg
   Mytomicin C
   Natalizumab
   Neosar
   Neovastat
   Nolvadex
   NV1020
   Oblimersen (Genasense)
   OK-432
   OL(1) p53
   Oregovomab
   OSI-774 (Tarceva)
   p53
   Panretin
   Perifosine
   Phenoxodiol
   Phenyl acetate
   Phenyl butyrate
   PI-88
   Pioglitazone
   Pivaloyloxymethyl butyrate
   PKC 412
   PKI 166
   PNU-145156E
   PNU-166196
   Prinomastat
   Propecia
   Proscar
   PS-341
   PSC 833
   PSK
   PTK/ZK 787
   PV701
   Pyrazoloacridine
   Quinine
   R-101933
   R115777 (Zamestra)
   Reolysin
   RhuMab-VEGF
   Rituximab
   RO 31-7453
   RPR/INGN-201
   Rubex
   SB-408075
   SCH66336
   SGN-15
   Squalamine
   SS1-PE38
   ST1571 (Gleevec)
   SU-101
   SU5416
   SU6668
   Suramin
   Swainsonine
   TAC-101
   Tamoxifen
   Taurolidine
   Tazarotene
   Temodar
   Temozolomide
   tgDCC-E1A
   Thalidomide
   Tirapazamine
   TK gene
   TLK286
   TNP-470
   TP-38
   Transretinoic acid
   Trastuzumab (Herceptin)
   Trelstar Depot
   TriGem
   Triptorelin
   Troglitazone
   Ubenimex
   UCN-01
   Vaccines
   Verapamil
   Vitxain
   WX-G250
   XR9576
   ZD1839 (Iressa)
   ZD6126
   ZD6474
   E7070
   Edrecolomab
   E1A-lipid complex (Targeted Genetics)
   GX01 (Gemin X Biotechnologies)
   Immunoconjugate antibody with toxin
   INGN201 (Introgen Therapeutics)
   ONYX-015 (Onyx Pharmaceuticals)
   SCH58500 (Schering-Plough)
   Suberoylanilide hydroxamic acid
   TRAIL (Genentech/Immunex)
   A5B7 with carboxypeptidase A

Suitable polymers for use in the inventive implant include, for example, those listed below specifically from the standpoint of different absorption and/or degradation rates:
Slowly absorbable/bioabsorbable/degradable polymers:
   polydioxanone, polyglycolide, polylactides [poly-L-lactide, poly-D,L-lactide and copolymers as well as blends such as poly(L-lactide-co-glycolide), poly(D,L-lactide-co-glycolide), poly(L-lactide-co-D,L-lactide), poly(L-lactide-cotrimethylene carbonate)], poly-ε-caprolactone, di- and triblock copolymers of the aforementioned lactides with polyethylene glycol, polyhydroxyvalerate, ethylvinyl acetate, polyethylene oxide, polyphosphorylcholine, polyhydroxybutyric acid (atactic, isotactic, syndiotactic as well as blends thereof), polyortho esters, polyanhydrides, etc.
Rapidly absorbable/bioabsorbable/degradable materials:
   fats, lipids (e.g., cholesterol, cholesterol esters and mixtures thereof), saccharides (alginate, chitosan, levan, hyaluronic acid and uronides, heparin, dextran, nitrocellulose, cellulose acetate and/or derivatives of cellulose, maltodextrin, chondroitin sulfate, carrageenan, etc.), fibrin, albumin, polypeptides and their derivatives, etc.

It is advantageous to achieve complete absorption of all components in the body. In many cases, in particular in palliative therapy, nonabsorbable materials may also be used. Preferred materials for individual components here are:
For carrier bodies such as stents or rods, tubes, grids:
   CoCr alloys
   Medical stainless steel 316L
   Nickel-titanium alloy
Coatings and/or materials of the nonabsorbable/permanent polymers:
   polypropylene, polyethylene, polyvinyl chloride, polyacrylate (polyethyl and polymethyl acrylate, polymethyl methacrylate, polymethyl-co-ethyl-acrylate, ethylene/ethyl acrylate, etc.), polytetrafluoroethylene (ethylene/chlorotrifluoroethylene copolymer, ethylene/tetrafluoroethylene copolymer), polyamide (polyamide imide, trogamide PA-11, PA-12, PA-46, PA-66 etc.), polyether block amide (Pebax with various hardeners), polyether imide, polyether sulfone (and blends), polyesters, polycarbonate, polyphenylsulfones (and blends), poly(iso)butylene, polyether ether ketone (PEEK) and blends thereof (with PES, for example), polyvinyl chloride, polyvinyl fluoride, polyvinyl alcohol, polyvinyl acetate, polyurethane (e.g., pellethane, elasthane), polybutylene terephthalate, silicones, polyphosphazenes, polyphenylene, polymer foams (e.g., from carbonates, styrenes, etc.), as well as copolymers and blends of the aforementioned classes and/or the class of thermoplastics and elastomers in general.

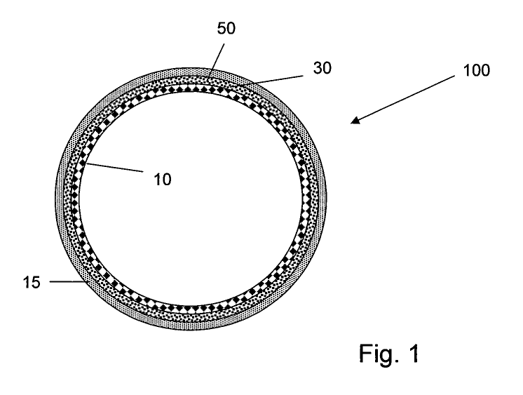

To illustrate the invention, FIG. 1 shows a cross section through an embodiment of a preferred drug-loaded implant 100 having a carrier body 10 and a drug 50 for delivery in a delivery region. The carrier body 10 is designed to be hollow and may be positioned in a blood vessel for example. The drug 50 may be delivered into the bloodstream and trans-ported to its site of action.

The drug-carrying implant 100 is preferably loaded with cytostatics, which would lead to serious adverse effects due to their nature and/or potency if administered systemically. Topical administration makes it possible to deliver to a site of action doses that could not be achieved systemically with a comparable potency. In addition, the drug delivery may take place in a controlled manner so that the drug 50 is made available over a sufficiently long period of time.

Elution of the drug may be based on the following benchmark values:
The most homogeneous possible delivery of the drug over at least 2 weeks;
The peak dose should be reached no later than 24 hours after administration;
The decline in dose after the end of the treatment period should be as steep as possible.

For example, a stent base body (preferably self-expanding) coated with a drug 50 embedded in a polymer matrix 30, as is known from the coronary or peripheral field, is used as the preferred carrier body 10, for example. The coated carrier body 10 is then mounted on a mandrel and is provided with an impermeable coating 15 only on the outside. Polymers such as parylene, PES, PTFE and others may be used as the impermeable coating. The coating 15 is applied by dissolving the polymer together with the drug 50 and applying it to the carrier body 10 by means of an immersion process or a spray coating. The coating 15 in the intended active state of the implant in a blood vessel protects the vascular wall from direct exposure to the drug 50. The bloodstream can transport the drug 50 to the actual site of action.

A suitable material for the carrier body 10 is fundamentally CoCr, 316L or Mg, but the preferred material is a nickel-titanium alloy (nitinol). The design for the carrier body 10 is based on a wall stent, where the implant 100 is advantageously self-expanding.

A drug 50 having cytostatic properties, preferably doxorubicin, epirubicin, cisplatin, mitomycin C (as an individual substance or in combination) is incorporated into degradable polymers, e.g., poly-L-lactides, poly-D-lactides, polyglycolides, polydioxanone, polycaprolactones and polygluconates, polylactide acid-polyethylene oxide copolymers, modified cellulose, collagen, poly(hydroxybutyrates), polyanhydrides, polyphosphoesters, poly(amino acids), poly(alpha-hydroxy acid) and/or combinations thereof.

Nondegradable polymers such as the following are also conceivable: silicones, polyurethanes, acrylates and/or methacrylates, polyethylene and ethylene copolymers (e.g., polyethylene-vinyl acetate), polysulfones, polyphenylsulfones or polyether sulfones, polyether ether ketones, polyphenyls (e.g., self-reinforced polyphenylene (e.g., Proniva™)).

Preferred production of an implant 100 may be performed with the following steps:

PLLA (PLLA=polylactide acid, e.g., L214 S) is dissolved in chloroform (1 g/L). The drug 50 is added in an amount by weight between 10 wt % and 60 wt %, preferably 30 wt % to 40 wt %, based on the polymer. The solution is sprayed using DES coating systems known from the state of the art. After tempering (storage at an elevated temperature and vacuum to remove the solvent) the layer composition amounts to 100-10,000 μg, preferably 100-2000 μg, preferably 600-900 μg. The choice of the polymer matrix 30 and specific loading with the drug 50 depend on the delivery kinetics desired for the substance to be eluted.

The drug-loaded carrier body 10 is placed on a mandrel (e.g., PTFE or silicone rubber) for deposition of the impermeable coating 15 and then is coated again with parylene or PES (see above).

In this way, 1.5 g/L PES may be dissolved in chloroform, for example, to then perform a dip coating or spray coating. Parylenes are applied by vapor deposition at room temperature and a reduced pressure. The impermeable coating 15 may advantageously have a layer thickness of 1 μm to 5 μm, preferably 4 μm to 5 μm.

Through the topically limited delivery of the drug 50, adverse effects may be minimized in an advantageous manner while at the same time achieving a high dose available topically.

The drug-carrying implant 100 is advantageously used for HCC treatment, preferably with drug delivery exclusively on the blood side. The implant 100 delivers the drug 50 to the blood, so that it is not necessary to place the implant 100 in immediate proximity to a tumor. The implant 100 may therefore be affixed a few centimeters upstream from a tumor, for example. The drug 50 then travels through the bloodstream to the destination site. The external impermeable coating 15 ensures that the drug 50, i.e., the medication, is not delivered to the tissue, which is usually healthy, in particular the vascular wall, where it would cause necroses and inflammation.

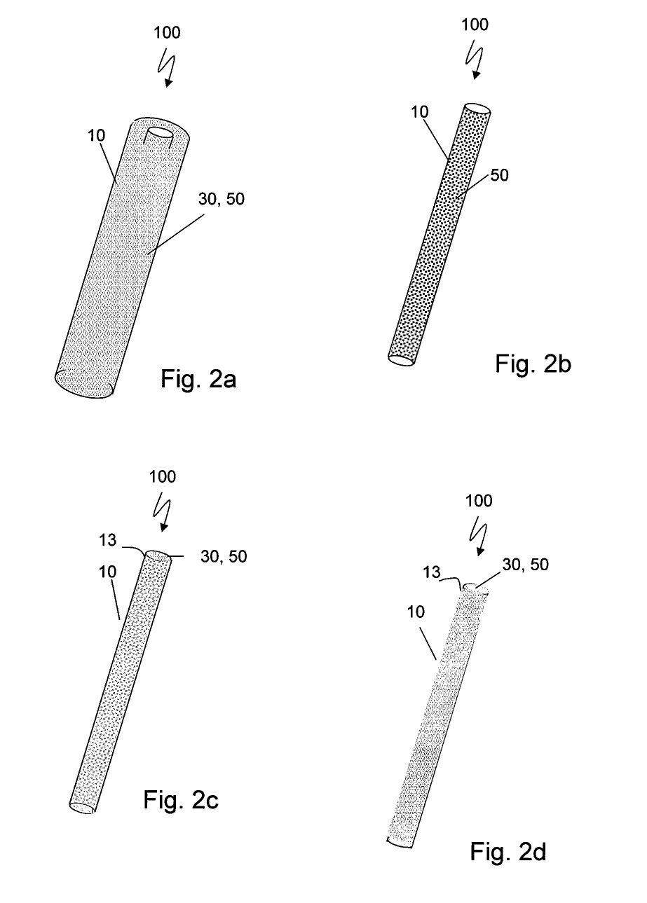

To illustrate another advantageous embodiment of the invention, FIGS. 2a to 2d show various preferred embodiments of a drug-loaded implant 100 having a carrier body 10 and a drug 50 for delivery in a delivery region, preferably in the body tissue in the form of a drug delivery pen. The drug 50 may be provided in particular for chemotherapy and/or palliative treatment and may be deliverable topically and/or regionally in a controlled manner at a predefinable rate and/or over a predefinable period of time when the carrier body 10 is inserted into the body tissue. The carrier body 10 may be coated or covered with a polymer or may comprise a polymer that is biodegradable and dissolves in the body into which the implant 100 has been applied. The implant 100 may optionally also comprise a nondegradable material or be formed from such a material.

Application of the implant 100 may also be accomplished through a suitably modified catheter or a trocar or the like. For example, FIG. 2 shows an advantageous device with which a plurality of implants 100 can be applied.

The carrier body 10 of the implant 100 may be hollow as shown in FIGS. 1a and 1b or may be solid as shown in FIGS. 1c and 1d and may preferably be positioned in the tissue. Drug 50 may be delivered topically to the tissue.

The drug-loaded implant 100 is loaded with cytostatics, for example, which would lead to serious adverse effects if administered systemically due to their type and/or efficacy. Topical administration makes it possible to deliver doses to a site of action that could not be achieved systemically with a comparable potency. In addition, the drug may be delivered in a controlled manner, so that the drug 50 is made available over a sufficiently long period of time.

Elution of the drug 50 may preferably be based on the following benchmark values, e.g., the most homogeneous possible delivery of the drug over at least 2 weeks; achieving a dose peak after no more than 24 hours; the steepest possible decline in dose after the end of the treatment period.

The invention makes it possible within the scope of topical therapy to administer one or more medications in a targeted manner in a high dose topically without inducing negative adverse effects in the surrounding tissue or body regions due to the mechanism of action and/or the dose of the medication.

The variants of the implant 100 illustrated in FIGS. 2a to 2d include a drug carrier, hereinafter referred to as the “drug delivery pen,” which advantageously makes it possible to administer a drug 50 in a very high dose topically or for regional forms of treatment as well as eliminating the need for a renewed procedure to remove the implant 100 because of its absorbable material character.

Another advantage of the preferred implant 100 embodied as a drug delivery pen consists of its design and/or its advantageous mechanical properties which allow the inventive implant 100 to be implanted in tissue for example even when it has a high drug content. In the state of the art, a high drug content usually reduces the mechanical properties of a carrier body greatly.

With the preferred implant 100, a reintervention to remove the implant may advantageously be avoided because of the absorbable materials. A high regional and topical delivery of a pharmaceutical drug is possible without causing adverse effects in healthy surrounding tissue and/or organs, for example.

The many exemplary variants for implementation of an implant 100 embodied as a drug delivery pen with a high drug load in order not to allow any damage to occur in implantation or during its lifetime, for example, are described below. The possible preferred choice of materials for the carrier body 10, the polymer matrices 30 and the drug 50 or drugs 50 was discussed in the introduction to the description.

FIG. 2a shows a first preferred variant of the preferred implant 100 embodied as a drug delivery pen. In this embodiment, the carrier body 10 of the implant 100 embodied as a drug delivery pen may comprise a solid-material rod or a tube. This carrier body 10 is sheathed with a drug-incorporated polymer layer 30 in which the drug 50 is embedded. The carrier body 10 may preferably be produced from bioabsorbable polymers and/or from bioabsorbable metals.

FIG. 2b shows a second preferred variant of a preferred implant 100 embodied as a drug delivery pen. As in the first variant, the carrier body 10 of the implant 100 embodied as a drug delivery pen may also comprise a solid-material rod or a tube. The second variant preferably comprises a polymer-free shoulder in which the rod/tube has either a roughened surface or a microstructured surface with voids (cavities) to be able to accommodate the drug 50 instead of carrying the drug 50 incorporated into the polymer as described in conjunction with FIG. 2a.

FIG. 2c illustrates a third preferred variant of an implant 100 embodied as a drug delivery pen. The third variant comprises a carrier body 10 formed from a tube having an inner cavity 13. The lateral surface of this tube may optionally be porous and/or permeable. A drug-loaded polymer matrix 30 is introduced as the core into the cavity 13 of the carrier body 10 designed as a tube. In the case of a permeable lateral surface, the drug 50 may travel outward from the core into the tissue in the cavity 13 of the carrier body 10 designed as a tube.

In the case of an impermeable carrier body 10 designed as a tube, the drug 50 is delivered in a targeted manner from the ends of the carrier body 10 of the implant 100 designed as a drug delivery pen. One end of the carrier body 10 embodied as a tube may optionally be closed here to allow a further increase in the targeted delivery of the drug.

FIG. 2d illustrates a fourth preferred variant of a preferred implant 100 embodied as a drug delivery pen. In this variant, the carrier body 10 of the implant 100 embodied as a drug delivery pen consists of a wire mesh, wire grid or stent, in whose inner cavity 13 is arranged a core made of a polymer with the drug 50 for example. The advantage of this metallic component of the carrier body 10 is that it guarantees the mechanical properties, as is also the case in the variants described above. In this specific case, it also represents protection of the body from fragments formed in the degradation and/or fragmentation of the drug-loaded polymer core. The drug 50 is preferably introduced into a drug-loaded polymer matrix in the cavity 13, as in the third variant.

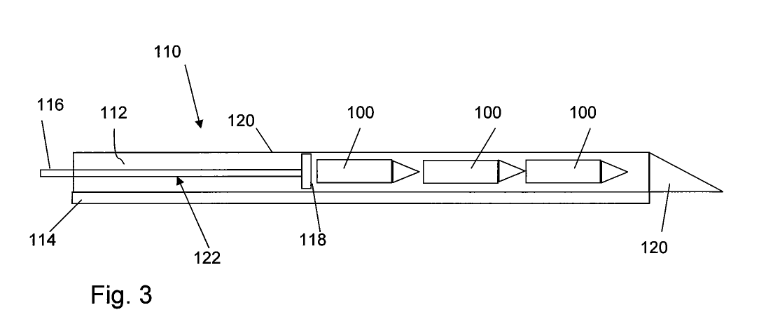

FIG. 3 illustrates an advantageous insertion system 110 for one or more implants 100 embodied as a drug delivery pen. Multiple implants 100 embodied as drug delivery pens may be arranged in series in an interior space 112 of the insertion system 110. Proximally from the most proximal implant 100 embodied as a drug delivery pen is situated a ram 118, which can be moved outside of the patient by means of a manipulator 116 by displacement against the outer shaft 120 of the insertion system 110 to displace the implants 100 distally out of the insertion system 110, each implant embodied as a drug delivery pen, situated in series. A distal tip 120 of the insertion system 110 embodied as a catheter, for example, may be embodied as a needle to simplify access to the target tissue. An insertion wire, which is known with catheters in general for facilitating administration may be provided in a guide 114 and may protrude beyond the distal tip 120 on insertion of the insertion system 110 into the body.

The advancing mechanism 122 expediently has a screen function, which enables the delivcry of a single implant 100 embodied as a drug delivery pen. In this way, one or more implants 100, each embodied as a drug delivery pen, can be delivered to multiple neighboring target regions. The insertion system 110 thus also offers protection for the implants 100, embodied as a drug delivery pen, from mechanical abrasion or loss of the drug to the surrounding bloodstream during positioning.

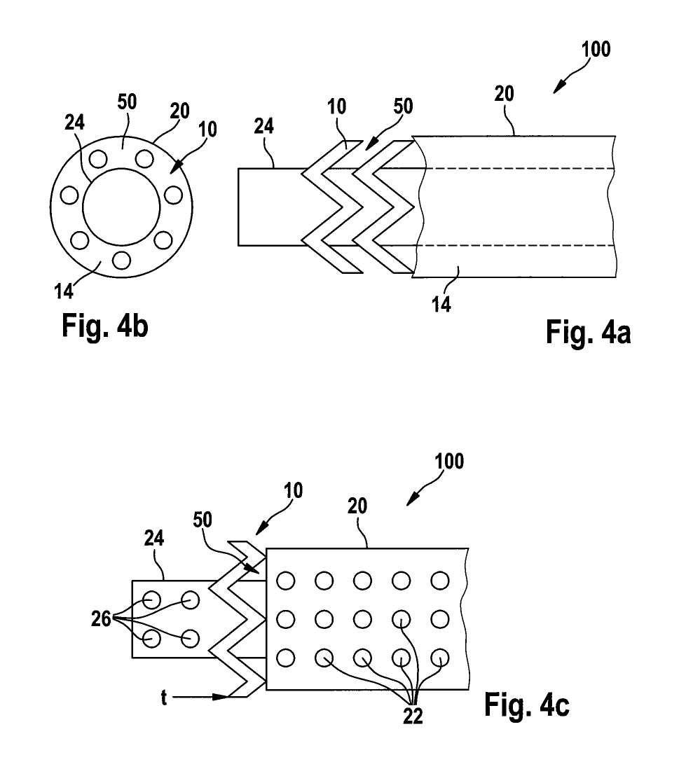

The existing concepts about the regional use of the drug through drug depots introduced arterially are often unable to introduce sufficiently large quantities of drug into defined regions. Another preferred exemplary embodiment of a preferred drug-loaded implant 100 is illustrated in two variants in FIGS. 4a to 4c, these being especially suitable for RDD (RDD=regional drug delivery). A multilayer implant 100 having an integrated stent as a carrier body 10 is preferred.

The preferred implant 100 is advantageously capable of delivering large quantities of a drug 50 (e.g., 5 mg to 100 mg, preferably 10 mg to 100 mg) into an arterial vessel. The implant 100 may be inserted through a catheter, e.g., into an arterial vessel.

The implant 100 consists of a covered stent (stent graft) as the carrier body 10, which is preferably self-expandable. This carrier body 10 is covered on both the inside and outside, e.g., by a polymer material 30 or polymer material systems, such as those described above in the introduction as an example. Between the outer cover 20 and the inner cover 24 of the carrier body 10, there is the drug 50 which is in a cavity 14 between the covers 20, 24, as shown in a partially cut-away view in FIG. 4a and in cross section in FIG. 4b. The function of the carrier body 10, which may preferably be made of nitinol (NiTi) is to affix the implant 100 to the vascular wall. The drug 50 may be delivered through the outer cover 20, through the inner cover 24 or through both covers 20 and 24 simultaneously. The preferred embodiments are those in which the drug 50 is not delivered to the vascular wall but instead is delivered exclusively to the lumen of the blood vessel into which the implant 100 is inserted to thereby avoid unwanted adverse effects in the vascular wall such as inflammation or necroses. The outer cover 20 here may be impermeable and the inner cover 24 may be designed to be permeable to suppress diffusion of the drug 50 through the outer impermeable cover 20 and/or to allow diffusion through the inner permeable cover 24.

Multiple variants are fundamentally conceivable. In a first variant, the covers 20 and/or 24 may be made at least partially of a degradable material, e.g., degradable polymer, fibrin, acrylic or fabric, whereby the drug 50 is delivered through degradation of the covers 20 and/or 24. In a second variant, the drug 50 may be delivered through perforated grafts, i.e., perforations in the covers 20 and/or 24. In a third variant, the carrier body 10 may be formed from degradable materials (degradable polymers or metals).

According to the first variant, the implant 100 is a self-expanding nitinol graft stent. The spaces between the outer adluminal cover 20 of the carrier body 10 are filled with the drug 50. The coating materials consist of biodegradable materials, for example, as in the examples listed above. Within the context of the process of degradation of the cover 20, the drug 50 is released. In a preferred embodiment, the drug 50 is delivered only luminally. The inner (ad)luminal cover 24 here is degradable, so that the drug 50 is released during the degradation process while the outer (impermeable) cover 20 is either not degradable or degrades so slowly that the degradation process begins significantly only after the drug 50 has already been delivered completely into the lumen.

In the second variant, the implant 100 constitutes a graft stent as in the first variant, but the drug 50 here is released through the structural perforations 22 and/or 26 of the covers 20 and/or 24 (e.g., in the direction of the vessel or into the bloodstream or in both directions). This is diagrammed in FIG. 4c. The spaces between the outer and inner covers 22 and 24 of the carrier body 10 are filled with the drug 50 as in the first variant. The covers 20, 24 may be permanent or degradable materials; in the case of the latter, the drug 50 (in contrast with the first variant) is not released by degradation but instead is released primarily through diffusion. The mural (outer) cover 24 may in turn preferably be designed to be impermeable so as not to burden the vascular wall unnecessarily.

A third and fourth variant (not illustrated) may be embodied like the first and second variants but the carrier body 10 consists of degradable materials, e.g., degradable polymers or metals. In this case, the covers 20, 24 preferably consist of degradable materials, forming a fully degradable hybrid.

This concept allows the implantation of large quantities of drug in certain regions of the arteries. Implantation can be achieved through a suitably modified insertion system (stent delivery system SDS).

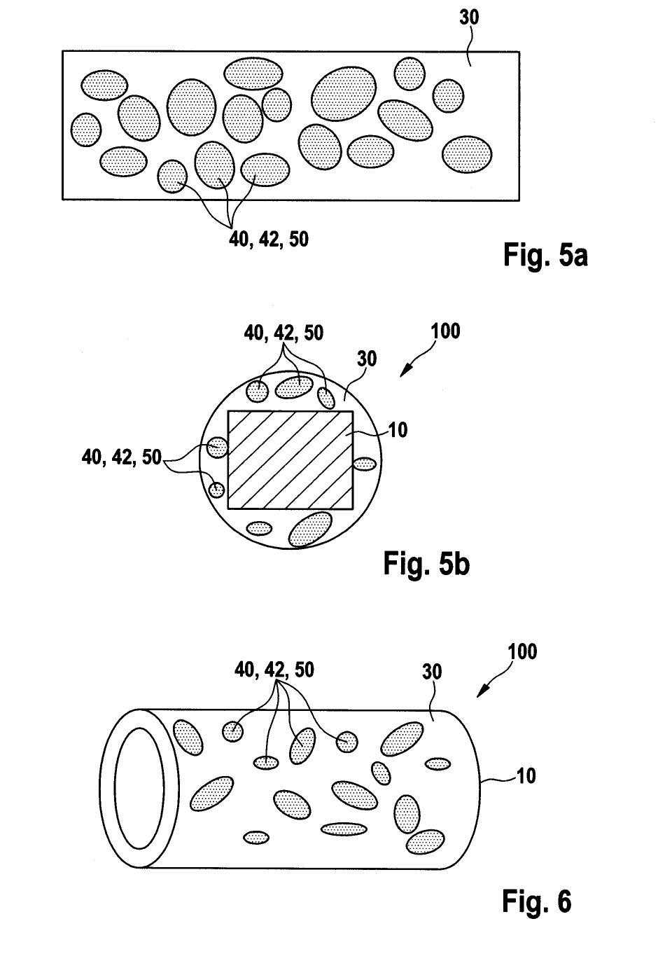

Another exemplary embodiment of the preferred implant 100 is illustrated in FIGS. 5a, 5b and FIG. 6 in two variants; this implant is especially preferably suitable for performing a TACE treatment using an RDD implant 100 (TACE=transarterial chemo-embolization) with additional potential for topical or regional release of a drug 50.

In conventional oncology, the TACE treatment represents a palliative procedure which essentially has a potentially curative approach in endocrinologic-oncologic treatment of neuroendocrine tumors. It is not limited merely to the elimination of symptoms that cannot otherwise be controlled (e.g., hypoglycemia). Liver metastases of a tumor are also supplied by arterial vessels of the liver. The hepatic arterial vascular system contributes approximately 30% to the oxygen supply, with 70% coming from the portal venous blood supply through the portal vein (vena portae). Therefore the liver tolerates a targeted “infarction” of the arterial blood supply through embolization of individual segments (partially selective) of an entire lobe of the liver or even the entire liver. Therefore, liver embolization has been used for many years with varying success for treatment of malignant primary diseases in oncology, e.g., in HCC (HCC=hepatocellular carcinoma). However, these are tumors that usually grow in an infiltrative and destructive manner, rapidly interfering with normal liver function. Metastases of neuroendocrine tumors, which usually grow slowly with a displacing (“pusher”) effect and hardly interfere with liver function at all, are almost always suitable for treatment by embolization therapies.

However, in the known embolization therapies due to the different materials used for embolization, but especially the fact that the proliferation tendency of the tumor vessels varies from one tumor to the next, the success of embolization in some cases lasts only a few weeks. Repeats are possible in the short term, are often required and are also indicated as a preventive measure when there are minimal findings. In addition to the risk of a time limit to the success of treatment by embolization, chemotherapy which is to be administered in another intervention step and in a high-dose single administration also constitutes a further burden for the patient and his health.

The preferred inventive implant 100 having a polymer matrix 30 advantageously combines the approach of TACE therapy, specifically the embolization, with a chemotherapy and/or medicinal therapy within the implant 100. Several medicines may be administered as the drug 50 separately over time and in variable doses regionally and topically; the size and rate of release of the embolization particles 40 causing the embolization are also controllable. With the TACE matrix, the embolization particles 40 may serve as carrier bodies for the medication. The embolization particles 40 are introduced into the vessel on a carrier body 10 and can be embedded within the embolization particles 40 more or less as an addon drug 50.

Through the interaction of the release of medication and the release of particles within just one procedure, a greater and longer-lasting therapeutic success is achieved. Thus, a decline in morbidity and reduced treatment costs can be achieved.

An inventive material matrix is preferred, preferably a polymer matrix 30, which is applied either to a carrier body 10 such as a stent as illustrated in FIG. 5b or can be introduced into a blood vessel without the use of a separate carrier, e.g., as tubing (FIG. 6), so that the polymer matrix 30 itself forms the carrier body 10. The preferred implant 100 advantageously combines the approach of TACE therapy (embolization) with chemotherapy and/or medicinal therapy within one component.

Thus, in the case of the present implant 100, the drug 50 is not administered intraarterially, but instead the drug 50 is incorporated into one or more of the polymers used in the TACE matrix (polymer matrix 30). This has the advantage that several medications can be administered as the drug 50 in accurate doses topically in a targeted manner and independently over time without thereby causing adverse effects.

Furthermore, with the preferred implant 100, it is possible to induce not just a single embolization, in which case a lasting therapeutic success is unclear, but instead to induce “sequential embolization” through controlled release, preferably over time and/or quantity, of embolization particles 40 which induce embolization. Repeated procedures can thus be reduced or, in the best case, even avoided completely.

With the implant 100, there is advantageously no separation between chemotherapy and embolization therapy. Accurate dosing and release of the embolization-inducing embolization particles 40 over time can be achieved in contrast with a single dose according to conventional therapy. This makes it possible to implement pharmaceutical therapy and/or chemotherapy as an adjunct to embolization in which targeted doses of medicine are delivered in a regionally and topically targeted manner over a longer period of time for treatment and thus do not lead to adverse effects such as those occurring with traditional forms of therapy using high-dose individual administrations. Through a suitable choice of materials, the implant 100 can be manufactured, so that it is completely absorbable over a large window of time, thus eliminating the need for an intervention to remove the implant 100.

One component of the implant 100 is the polymer matrix 42 (see schematic diagram in FIG. 5a), which represents the embolization particles 40 to be released for embolization in the preferred sizes between 2 μm and 200 μm. These embolization particles 40 are incorporated into the polymer matrix 30. The embolization particles 40 can be produced by spraydrying processes, for example. The embolization particles 40 may be formed from degradable or permanent polymers or may comprise a superabsorbent material.

A surface modification of the embolization particles 40 with the goal of suppressing chemical bonding to the material of the polymer matrix 30 is optional and may be evaluated according to the polymer pairings used for the matrix 42, the embolization particles 40 and the polymer matrix 30. The matrix 42 may also be omitted and the embolization particles 40 may be embedded directly in the polymer matrix 30.

Depending on the degree of filling of the polymer matrix 30 with the first matrix 42, the number of embolization particles 40 that induce embolization can be controlled. The dosage and thus also the treatment time of embolization can be adjusted through the rate of absorption and degradation of the polymer matrix 30.

In addition to release of pure particles, both matrices 30, 42 may be loaded with drug 50 to support the treatment. Specifically through embolization particles 40 loaded with drug 50, the drug 50 can be administered in a highly topical manner.

A favorable variant in FIG. 5b shows the TACE matrix consisting of polymer matrix 30 and embolization particles 40 and/or matrix 42 on a stent as the carrier body 10. In this variant, the inner luminal stent side is ideally in coded form and can be implemented easily by technical modifications of the process in production.

Furthermore, it is also possible to position the TACE matrix between two stents as the carrier bodies 10 (not shown).

In a second variant, the TACE matrix is glued or pressed as a self-supporting tube into a vascular wall, so that the polymer matrix 30 forms the carrier body 10 itself. An application may be implemented by means of a balloon catheter with an adhesion promoter for adhesion with the vascular wall, e.g., fibrin, acrylates, being conceivable. Applying a “protective sheathing” over a delivery stent comparable to the sheathing with self-expandable stents is advisable to allow implementation of positioning and/or fixation and/or adhesion of the stent at the desired location in the delivery region. This tubing may of course also optionally be applied to a stent as the carrier body 10.

A list of suitable polymers for use as the TACE RDD matrix, as already mentioned with the other exemplary embodiments, is given in the introduction to the description, specifically from the standpoint of different absorption rates and/or degradation rates.

Whereas the choice of materials listed under the heading “rapidly bioabsorbable/degradable materials” and under the heading “slowly absorbable/bioabsorbable/degradable polymers” has the aim of creating a temporary closure of the supplying vessels (temporary embolization), suitable permanent polymers (see heading “permanent polymers”) may also be selected or the embedded embolization particles 40 may also consist of a so-called superabsorbable material which swells in aqueous systems after being released.

Favorable superabsorber materials include, for example, typical materials such as polyethylene oxide, polyvinyl alcohol, polyacrylic acid (crosslinked, partially crosslinked; partially neutralized), polyacrylate, polymer blends of polyacrylic acid and sodium acrylate, nonionic polymers (e.g., crosslinked polyacrylamide), polycarboxylates, polycyanoacrylate, polyvinylbutyral, etc. In general, the class of superabsorbers is understood to include crosslinked or partially crosslinked as well as surface-crosslinked or bulk- and corecrosslinked polymers or polymer blends. Typical crosslinking agents here include for, example, tetraallylethoxyethane or 1,1,1-trimethylolpropane triacrylate (TMPTA).

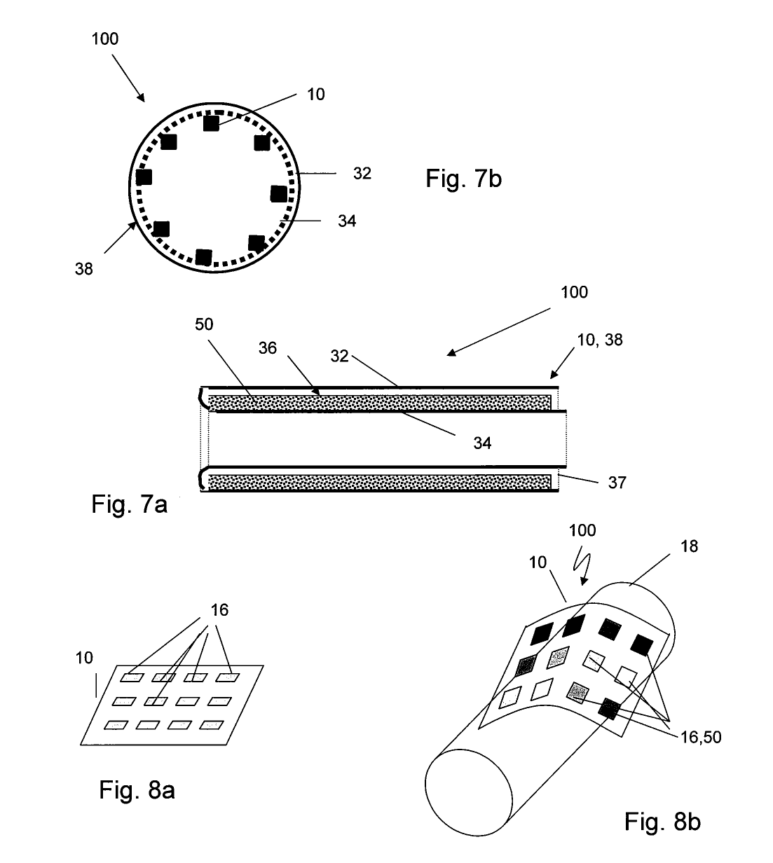

Another advantageous refinement of the invention is illustrated in FIGS. 7a and 7b. The preferred implant 100 in this embodiment comprises a tubular material, which can be inverted and can hold the drug 50 in the cavity thereby formed. This refinement is especially preferably used in the treatment of inoperable HCC.

The drug-loaded implant 100 may be loaded with cytostatics as the drug 50, which would lead to serious adverse effects if administered systemically due to their nature and/or potency. Topical administration makes it possible to deliver doses that could not be achieved systemically with a comparable potency and to introduce them to the site of action. In addition, the drug delivery may take place in a controlled manner so that the drug 50 is made available over a sufficiently long period of time. Elution of the drug 50 can be based on the following benchmark values:
the most homogeneous possible delivery of the drug 50 over at least 2 weeks;
the peak dose should be reached after 24 hours at the latest;
the decline in dose after the end of the treatment period should be as steep as possible.

A preferred tubular implant 100 can be produced with the following steps. The implant 100 may be self-supporting or may have a carrier body 10 as the supporting structure, e.g., of nitinol or other metals or polymer materials.

The tubing 38 consists of a flexible polymer material and it is half-wrapped (FIG. 7a), thus forming an outer tubular section 32 and an inner tubular section 34. The cavity 36 between the tubular sections 32, 34 is used as a drug reservoir for the drug 50. With this approach, it is necessary to produce a 36-mm-long tubing 38, for example, for an 18-mm-long tubing implant 100.

As an alternative, the tubing 38 may be extruded as a coaxial double tube with an inner tubing (corresponding to the tubing segment 34) and an outer tubing (corresponding to the tubing segment 32). In addition, a coextrudate may be applied to the inner tubing via a ring gap. The drug 50 can be introduced into the polymer melt of the inner tubing. The inner tubing then forms a drug-loaded polymer matrix 30. FIG. 7b shows a cross section through such a coextruded tubing 38. For example, a stent may be provided in the interior of the tubing 38 as the carrier body 10.

The implant 100 delivers only the drug 50 to the bloodstream via the tubing 38 through which the blood flows and does not deliver any drug to the vascular wall or the tissue at the implantation site. A number of preferably thermoplastic materials are conceivable as the tubing material.

The implant 100 may also be formed in the shape of a spiral (not shown). The spiral consists of a tube, which is filled with the drug or with a drug formulation. Nitinol is especially suitable as the material. The spiral remains in the vessel, with the drug being delivered through the opening. The drug delivery can be influenced by notches provided over the length of the spiral.

Drugs with cytostatic properties that can be used for the implant preferably include doxorubicin, epirubicin, cisplatin, mitomycin C (as an individual substance or in combination) or other suitable listed drugs 50.

The drug 50 may be used as a pure substance or as a formulation. The following polymers may be used to achieve a suitable drug delivery kinetics: polylactides such as poly-Llactides, poly-D-lactides, polyglycolides, polydioxanone, polycaprolactones, poly-(hydroxybutyrate) and polygluconates, polylactide acid-polyethylene oxide copolymers, saccharides such as modified cellulose, alginate, chitosan, polypeptides such as collagen or Matrigel®, polyanhydrides, polyortho esters, poly(alpha-hydroxy acid) and combinations thereof.

Likewise, nondegradable polymers may also be used such as silicones, polyurethanes, acrylates and/or methacrylates, polyethylene and ethylene copolymers, such as polyethylene vinyl acetate, polysulfone, polyphenylsulfones or polyether sulfones, polyether ether ketones, polyphenyls, such as self-reinforced polyphenylene (e.g., Proniva™).

Liposomal encapsulation or microencapsulation are also suitable for a targeted use of the drug delivery behavior. Furthermore, cyclodextrins, in particular beta-cyclodextrin or 6-O-palmitoyl-L-ascorbic acid, may also be used as an additive when using polymers from the list above.

An advantageous production process for the tube 38 may be carried out using the following steps, for example (without other materials or compositions, other process parameters may also apply):

polyethylene glycol (molecular weight 4000 g/mol) is ground finely in a mortar and dried for 5 days over phosphorus pentoxide in a desiccator under a reduced pressure (water jet vacuum, 17 mmHg). 1 g PEG (polyethylene glycol) is weighed into each 2 mL Eppendorf test tube and melted at 62° C.; then 100-200 μL HMDI (hexamethyl diisocyanate) is added to the melt. The two-phase mixture is thoroughly mixed using a vortexer and incubated for 1.5 hours at 62° C. The pot life is 30 minutes. The melt is stirred with a Pasteur pipette. The material is processed from the melt based on the pot life.

To do so, templates (glass or metal rods having the desired inside diameter) are coated with the melt and stored for 12 hours at room temperature. After the storage time has elapsed, the tubes are released from the template. The release is facilitated if the tubes 38 can swell in double-distilled water. These are tubes 38 having a homogeneous wall thickness and homogeneous dimensions which can be cut to size and sterilized. Steam sterilization is possible.

These tubes 38 can be half-folded (FIG. 7a). The resulting cavity between the outer and inner tube segments 32, 34 can be filled with the drug 50. With the opening 37 at the distal end, the implants 100 are advanced into the blood vessel to the delivery region.

A coextruded tubing 38 (FIG. 7b) with a drug-loaded inner tubing 34 over which an impermeable outer tubing 32 is applied by coextrusion can be produced with the following steps.

A drug solution containing the drug 50 is introduced into the tubing, which is to form the inner tubing segment 34. After evaporation of the solvent, the tubing has an inside surface lined with the drug 50. To influence the release of the drug, the solution may also contain a polymer or an agent from the list given above in this exemplary embodiment or the list presented at the introduction with the help of which delayed release can be achieved. The method mentioned last stands out due to its simple design and may therefore be preferred.

Due to the topically limited release of the drug 50, adverse effects are minimized, while at the same time achieving a high dose available topically. The implant 100 delivers the drug 50 to the bloodstream, so that it is not necessary to place the implant 100 in immediate proximity to a tumor. The implant 100 may therefore be affixed a few centimeters upstream from the tumor. The drug 50 then arrives at the destination site via the bloodstream. The outer sheathing ensures that the medicine is not delivered to the tissue, in particular the vascular wall, which is not usually diseased and where it could cause necroses and inflammation.

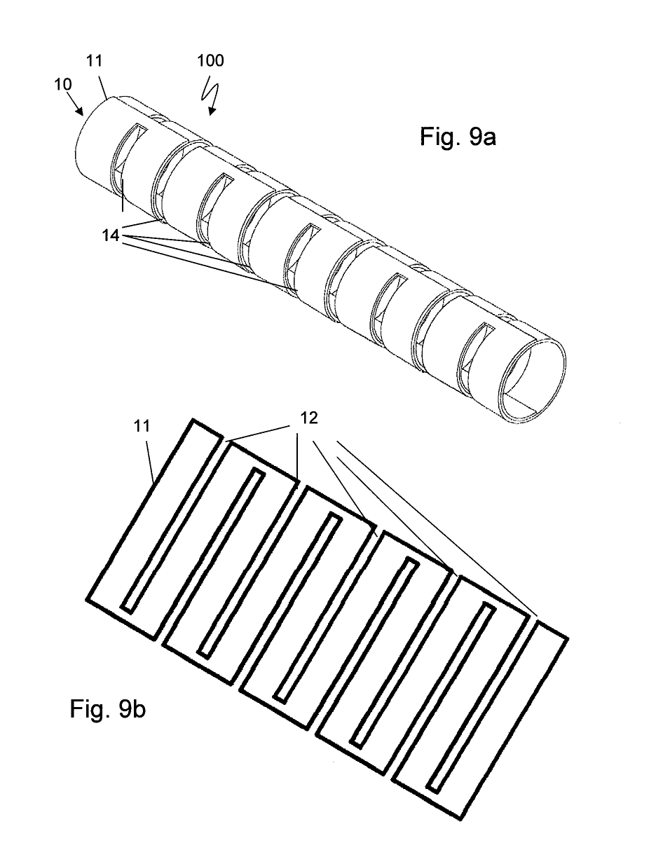

FIGS. 8a, 8b and 9a, 9b show variants of another preferred embodiment of an inventive drug-loaded implant 100.

FIGS. 8a and 8b show an drug 50 embedded in a film 11 as a carrier body 10, which may be formed, e.g., from nitinol or other materials, e.g., stainless steel 316L, CoCr, Mg. The film 11 preferably has a thickness between 40 μm and 1000 μm, especially preferably 200 μm, and is provided with recesses 16 by means of a deep-drawing process, the drug 50 being introduced into said recesses as a pure substance or as a formulation with the polymers mentioned above and/or in the introduction or other auxiliary materials such as polyvinylpyrrolidone, PEG (polyethylene glycol), cyclodextrins, PVA (polyvinyl alcohol) in different degrees of saponification, hydrogels such as hyaluronic acid, alginate, chitosan, gelatins, 6-O-palmitoyl-L-ascorbic acid or lauric acid.

The film 11 is rolled around a balloon 18 (FIG. 8b) with the recesses 16 for intruding inward and is affixed with a protective tubing. After being positioned in the blood vessel, the protective tubing is retracted and the film 11 with the drug 50 is thus released.

A drug 50 having cytostatic properties is preferred, e.g., doxorubicin, epirubicin, cisplatin, mitomycin C (as a single substance or in combination) or materials that are mentioned in the introduction and can be introduced into degradable polymers, e.g., poly-L-lactide, poly-D-lactide, polyglycolide, polydioxanone, polycaprolactone and polygluconate, polylactide acid-polyethylene oxide copolymers, modified cellulose, collagen, poly(hydroxy-butyrate), polyanhydride, polyphosphoesters, poly(amino acids), poly(alpha-hydroxy acid) and combinations thereof.

Nondegradable polymers may also be provided, e.g., silicones, polyurethanes, acrylates and/or methacrylate, polyethylene and ethylene copolymers such as polyethylene vinyl acetate, polysulfones, polyphenylsulfones or polyether sulfones, polyether ether ketones, polyphenyls such as self-reinforced polyphenylene (e.g., Proniva™).

For example, a film 11 approximately 200 μm thick with a length of 18 mm, for example, and a width of 13 mm, for example, is provided with small wells or recesses 16 in a punching device. Favorable dimensions for the film 11 are in a range from 8 mm to 30 mm in length, for example, and in a range of 4 mm to 30 mm in width, for example. The recesses 16 preferably have dimensions from 1 mm to 2 mm in length and 1 mm to 2 mm in width, or a corresponding diameter in the case of a round embodiment. The film thickness is preferably in the range between 60 μm to 300 μm.

The drug 50 is introduced into the recesses 16, e.g., by dipping and stripping off the surface, spray coating or ink-jet methods, in which the holes are filled separately, impressing drug beads or pressed items.

FIGS. 9a and 9b show a variant in which the film 11 is provided with slots 12 (FIG. 9a) and is rolled up. Drug 50 may be introduced into the cavities 14 formed by the slots 12 or the side that is to form the inside of the rolled-up film 11 is coated with the drug 50 in pure form or in a polymer matrix (not shown). It is also conceivable for a drug delivery pen (see FIGS. 2a-2d) to be introduced into the interior of the rolled-up film 11. The rolled-up film 11 forms a carrier body 10 for the drug 50. The film 11 may also be formed from a degradable material.

Due to the topically limited release of the drug 50, adverse effects are minimized, while at the same time achieving a high topically available dose.

The implant 100 delivers the drug to the bloodstream, so that it is not necessary to place the implant 100 in immediate proximity to the tumor. The implant 100 may therefore be affixed a few centimeters upstream from the tumor. The drug then reaches the destination site through the bloodstream. The design of the implant 100 ensures that the drug 50 is not delivered to the tissue, which is usually healthy and where it could cause necroses and inflammations.

1. A drug-loaded implant having a carrier body and at least one drug for release into a delivery region, characterized in that the drug is provided for at least one of chemotherapy and palliative treatment, and wherein the implant is characterized in that the drug can be released in the body of a living creature in at least one of a predefinable rate and over a predefinable period of time, and characterized in that the drug release process is controlled in at least one of topically and regionally in the intended active state of the implant.

2. The implant according to claim 1, characterized in that the carrier body biodegradable.

3. The implant according to claim 1, characterized in that the drug is embedded in a polymer matrix from which the drug can be released by degradation of the polymer matrix.

4. The implant according to claim 3, characterized in that the carrier body is coated with the polymer matrix in at least some areas.

5. The implant according to claim 1, characterized in that the drug is provided in at least one of recesses and cavities in the carrier body.

6. The implant according to claim 1, characterized in that the polymer matrix forms the carrier body in at least some areas.

7. The implant according to claim 1, wherein the carrier body is hollow, and characterized in that the drug is arranged between two polymer layers which surround the hollow carrier body adluminally and abluminally.

8. The implant according to claim 1, characterized in that the carrier body is formed by a tubing, which is inverted in some areas, whereby the drug is arranged in the area of a cavity formed by the inversion.

9. The implant according to claim 1, characterized in that embolization particles to be released in a polymer matrix are provided.

10. The implant according to claim 9, characterized in that at least one of the polymer matrix and the embolization particles are loaded with the drug.

11. The implant according to claim 1, characterized in that a drug-impermeable cover layer is provided, protecting at least one of the tissue and vascular walls of the delivery region from the drug in the implanted state.

12. The implant according to claim 1, characterized in that the carrier body is embodied as a stent.

13. The implant according to claim 1, characterized in that the carrier body has the general shape of at least one of a tube and a rod.

14. The implant according to claim 1, characterized in that the carrier body is formed from a rolled film having recesses to receive the drug.

15. The implant according to claim 1, characterized in that the carrier body (10) is formed from a rolled and slotted film.
